(19) United States
(12) Patent Application Publication
(10) Pub. No.: US 2024/0099757 A1
(43) Pub. Date: Mar. 28, 2024

(54) HELIUM PLASMA NUTATIONAL INFRASONIC LIPOSCULPTURE SYSTEM AND METHOD

(71) Applicant: EBS Enterprises LLC, Miami Beach, FL (US)

(72) Inventor: Aaron J. ROLLINS, Miami Beach, FL (US)

(21) Appl. No.: 17/954,803

(22) Filed: Sep. 28, 2022

(51) Int. Cl. A61B 18/04; A61B 17/32; A61B 17/34; A61M 1/00; A61M 5/30; A61M 11/00

(52) U.S. Cl. CPC A61B 18/042; A61B 17/320068; A61B 17/34; A61M 1/89; A61M 5/30; A61M 11/007; A61B 2018/00464; A61M 2202/048; A61M 2202/08

(57) ABSTRACT

A minimally invasive body sculpting system and method is provided using a numbing component, a helium plasma component, and an NIL component. An NIL cannula of the NIL component is insertable into the body to remove the fat cell via nutational movement thereof and suction there through. Skin tightening is achieved through the subcutaneous exposure of the skin to helium plasma.

### FIELD OF THE INVENTION

The invention relates to surgical procedures. More particularly, the invention relates to body sculpting surgical procedures.

### BACKGROUND

Liposuction is a cosmetic surgery operation that removes fat from a site within a body. Typically, liposuction may be performed to remove fat from the abdomen, thighs, buttocks, neck, back, arms, and other parts of the body. Early liposuction procedures used curettage techniques, which involved scraping or scooping the fat from within the body. These techniques were abrasive and often involved health risks.

Due to the high risks involved with curettage, liposuction techniques evolved to use a suction-assisted removal after infusing a liquid into tissue. However, this method achieved only mixed results and raised concerns over effects caused to the body from the introduction of a high volume of fluid. Also, concerns arose regarding potential toxicity of these fluids when introduced into the body at high volume.

To cope with these concerns, physicians began using ultrasonic techniques to liquefy fat for removal. These techniques involved focusing ultrasonic energy at the fat, causing it to become a liquid that could be removed. However, due to an increased report of complications, such ultrasonic methods have also raised concerns. Additional techniques that differentiate from general liposuction have been created. For example, nutational infrasonic liposculpture (NIL) is a technique that implements a system in which infrasonic vibration is used to remove fat.

Certain liposculpture procedures have evolved to also use lasers. Lasers heat the fat, making it easier to remove, and also heat the surrounding tissue providing a degree of skin tightening after the procedure. However, using lasers with currently known liposculpture procedures may not provide as much skin tightening as may be desired. A more recent alternative to laser skin tightening is skin tightening through subcutaneous helium plasma, for example, the RENUVION treatment made available by Apyx Medical Corporation. While just recently obtaining US Food and Drug Administration approval for limited uses, such helium plasma treatments have also been found to have the potential for significant injury. There is a need for safer body sculpting procedures that are efficient at removing fat and tightening skin to improve the overall results.

### SUMMARY

According to embodiments of the present invention, a helium plasma NIL system and method is described that may remove fat from within a human. The helium plasma NIL technique, according to an embodiment of the present invention, may remove fat from within a person using a method with high efficiency and effectiveness. The helium plasma NIL method of the present invention also provides a procedure that is minimally invasive and causes little to no pain.

The present invention advantageously allows a patient to reduce the volume of fat in his or her body using a relatively painless technique that produces results. Due to the minimally invasive nature of the methods and system, according to the embodiments of the present invention disclosed herein, a number of patients may experience an accelerated recovery and a decreased amount of downtime required for the body to heal.

Another advantage of the systems and methods described herein is that the incision is created by a biopsy punch. The NIL component and helium plasma component, in combination with an anesthetic component, does not use a needle and does not require stitches so that the method produces a naturally healing, scarless wound at the incision.

Still another advantage of the systems and methods described herein is that the procedure yields a high-quality fat that does not contain debris. The high-quality fat can be transferred to other areas of the body for use in other medical procedures such as, for example, cosmetic surgical procedures.

A method is provided by the present invention that improves on the fat removal techniques of the prior art. The method may include driving a numbing solution into a patient using a jet injector, eliminating the need for a needle. The method also advantageously provides access to the interior of a patient by using a biopsy punch, for example, a two-millimeter biopsy punch, eliminating the need for a scalpel. Through these advantages, the method improves over the prior art by conditioning a patient for efficient fat removal while eliminating the need for placement of stiches or other sutures.

The method provided by the present invention additionally improves on the prior art by numbing a patient using a cannula, such as a blunt 18-gauge cannula. Using the method of the present invention, the fat may be removed using cannulae from between 2 mm-4.5 mm that utilizes a powered infrasonic nutational system. The method of the present invention increases efficiency of fat removal from a patient while reducing the pain and recovery times caused by fat removal procedures of the prior art.

According to an embodiment of the present invention, a minimally invasive fat cell removal system is provided with a helium plasma component and an NIL component. The NIL component may remove the fat cell and the helium plasma component may tighten the skin. The skilled artisans will appreciate recitation of a fat cell throughout this disclosure to mean one or more fat cells, without limitation. The NIL component may include an NIL cannula and a suction fixture. The NIL cannula and the helium plasma cannula may be inserted into a body of a patient at an incision created by a biopsy punch. The fat cell that is at least partially liquefied is removable from the body via the NIL cannula.

In another aspect, the fat cell removal system may include an anesthetic device to provide reduced sensation of the body at a location, the anesthetic device including a numbing component. The cannula may be inserted at the location with reduced sensation. The anesthetic device may introduce a numbing compound via jet injection.

In another aspect, a tumescent fluid may be injectable via a tumescent cannula. The tumescent fluid can cause fat cells to become tumescent, prior to extraction. Injection of the tumescent fluid may facilitate the at least partial liquefaction of the fat cell. The fluid introduced through a separate tumescent cannula may introduce an anesthetic to a broader subcutaneous region beyond the immediate vicinity of the biopsy punch hole to reduce discomfort from the procedure.

In another aspect, the NIL component may provide nutational movement to the NIL cannula during removal of the fat cell. In another aspect, the NIL component may include an output through which the fat cell is harvestable. The fat cell that is harvested from the body from a first location may be implantable into the body at a second location to increase a volume of fat cells near or adjacent to the second location. In another aspect, a stem cell may be implantable with the fat cell at the second location to increase a likelihood of acceptance of the fat cell by the body at the second location.

According to an embodiment of the present invention, a method is provided for removing a fat cell. The method may include (a) inserting a cannula coupled to an NIL into a body of a patient at an incision generated by a biopsy punch, (b) using an NIL component to remove the fat cell and extracting the fat cell via suction through the NIL cannula, and (c) using a helium plasma component to tighten skin tissue proximate the region from which the fat cell was removed.

In another aspect of the method, before step (a), an additional step may be included for using a numbing component to reduce sensation of the body at a location using an anesthetic device. The anesthetic device may operate via jet injection.

In another aspect of the method, injecting a tumescent fluid via the tumescent cannula can cause the fat cell to become tumescent and facilitate the at least partial liquefaction of the fat cell. The tumescent fluid may also include a local anesthetic.

In another aspect of the method, the NIL component may provide nutational movement to the NIL cannula during removal of the fat cell.

In another aspect of the method, the NIL component may include an output through which the fat cell is harvestable. Step (b) may further include (d) harvesting the fat cell from a first location using the output.

In another aspect of the method, an additional step may be included for implanting the fat cell that is harvested into the body at a second location to increase a volume of fat cells adjacent to the second location.

In another aspect of the method, step (e) may further include implanting a stem cell with the fat cell at the second location to increase a likelihood of acceptance of the fat cell by the body at the second location.

According to another embodiment of the present invention, a method is provided for removing a fat cell that is minimally invasive. The method may include (a) using a numbing component to reduce sensation of a body of a patient at a first location using an anesthetic device. The method may also include (b) using an NIL component to remove the fat cell. Step (c) may further include inserting an NIL cannula into the body of the patient at the first location, and extracting the fat cell by suction through the NIL cannula, the fat cell being harvestable from the first location. Step (d) may include using a helium plasma component to tighten skin tissue proximate the location of the body from which the fat cell was removed.

In another aspect of the method, step (a) may further include creating a biopsy punch at the first location to create an incision through and into which the NIL component and helium plasma device are insertable.

In another aspect of the method, the incision created by the biopsy punch does not require stitches so that the method produces a naturally healing, scarless wound at the incision. In another aspect of the method, the NIL component may provide nutational movement to the NIL cannula during removal of the fat cell.

In another aspect of the method, the NIL component may include an output through which the fat cell is harvestable. After step (c), the method may further include step (d) harvesting the fat cell from the first location using the output.

In another aspect of the method, after step (d), an additional step may be included for step (e) implanting the fat cell that is harvested into the body at a second location to increase a volume of fat cells adjacent to the second location.

In another aspect of the method, step (e) may further include implanting a stem cell with the fat cell at the second location to increase a likelihood of acceptance of the fat cell by the body at the second location.

Unless otherwise defined, all technical terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control.

### BRIEF DESCRIPTION OF THE DRAWINGS

FIG. 1 is a block diagram of a helium plasma NIL system, according to an embodiment of the present invention.

FIGS. 2A-2C depict side, front, and back views of a biopsy punch.

FIG. 3 depicts a perspective view of one embodiment of a numbing component that is a jet injector.

FIG. 4 depicts a side view of the NIL component.

FIG. 5 depicts a tumescent cannula that can be used to inject tumescent fluid into an incision made by the biopsy punch.

FIG. 6 depicts an exterior view of the NIL component being inserted under the skin.

FIG. 7 depicts a cross-sectional view of the helium plasma component inserted under the skin of a patient.

FIGS. 8A-8F are patient photographs depicting before treatment and after treatment of the laser NIL and helium plasma NIL method.

FIG. 9 is flow chart describing a helium plasma NIL method, according to an embodiment of the present invention.

### DETAILED DESCRIPTION

The present invention is best understood by reference to the detailed drawings and descriptions set forth herein. Embodiments of the invention are discussed below with reference to the drawings; however, those skilled in the art will readily appreciate that the detailed description given herein with respect to these figures is for explanatory purposes as the invention extends beyond these limited embodiments. For example, in light of the teachings of the present invention, those skilled in the art will recognize a multiplicity of alternate and suitable approaches, depending upon the needs of the particular application, to implement the functionality of any given detail described herein beyond the particular implementation choices in the following embodiments described and shown. That is, numerous modifications and variations of the invention may exist that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

The present invention should not be limited to the particular methodology, compounds, materials, manufacturing techniques, uses, and applications described herein, as these may vary. The terminology used herein is used for the purpose of describing particular embodiments only, and is not intended to limit the scope of the present invention. As used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” may be a reference to one or more steps or means and may include sub-steps and subservient means.

All conjunctions used herein are to be understood in the most inclusive sense possible. Thus, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should be read as “and/or” unless expressly stated otherwise. Structures described herein are to be understood also to refer to functional equivalents of such structures. Language that may be construed to express approximation should be so understood unless the context clearly dictates otherwise.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “having” should be interpreted as “having at least”; the term “includes” should be interpreted as “includes but is not limited to”; the term “example” is used to provide exemplary instances of the item in discussion, not an exhaustive or limiting list thereof; and use of terms like “preferably,” “preferred,” “desired,” “desirable,” or “exemplary” and words of similar meaning should not be understood as implying that certain features are critical, essential, or even important to the structure or function of the invention, but instead as merely intended to highlight alternative or additional features that may or may not be utilized in a particular embodiment of the invention.

Those skilled in the art will also understand that if a specific number of an introduced claim recitation is intended, such an intent will be explicitly recited in the claim, and in the absence of such recitation, no such intent is present. For example, as an aid to understanding, the appended claims may contain usage of the introductory phrases “at least one” and “one or more” to introduce claim recitations; however, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C” is used, in general, such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.).

All numbers expressing dimensions, quantities of ingredients, reaction conditions, and so forth used in the specification are to be understood as being modified in all instances by the term “about” unless expressly stated otherwise. Accordingly, unless indicated to the contrary, the numerical parameters set forth herein are approximations that may vary depending upon the desired properties sought to be obtained.

The present invention will now be described in detail with reference to embodiments thereof as illustrated in the accompanying drawings. In the following description, a helium plasma NIL system and method will be discussed.

Additionally, the helium plasma NIL method may be performed using a helium plasma NIL system. At least one embodiment of the helium plasma NIL system will be described throughout this disclosure.

According to the various embodiments of the present invention, a medical procedure may be performed using the system disclosed herein to reduce the volume of fat in the body of a patient. The procedure may be performed using no needle or scalpel. As a result, the procedure may also not require stiches or removal of the same. Implementing this lower energy procedure yields a reduction in bruising as comparison to other treatments. Furthermore, the fat may be harvested with minimal debris and used for transfer to other parts of a patient's body.

Fat removal using the system and method of the present invention may be less invasive than standard liposuction and therefore there is less downtime and faster results. Whereas typical liposuction takes about 6 months to heal, the present invention may provide results in about a month in some situations. Being less invasive also means less pain after performance of the procedure. Many patients of the procedure, performed in accordance with the system and methods of the present invention, can be back at work sooner than with traditional fat removal methods of the prior art. The systems and methods disclosed herein further provide for improved skin tightening with increased safety in relation to other NIL treatments or helium plasma skin tightening treatments.

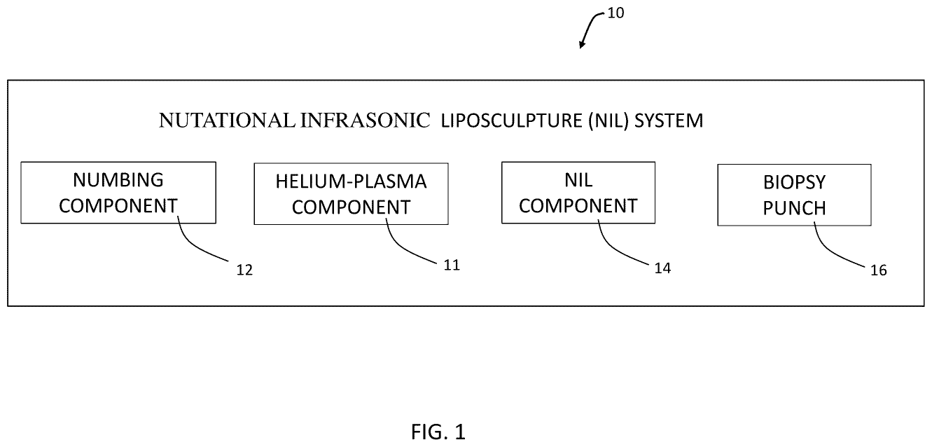

Referring now to FIG. 1, a helium plasma NIL system 10, according to an embodiment of the present invention, will now be discussed. Additionally, a method of using a helium plasma NIL system will be discussed. The helium plasma NIL system 10 may include a numbing component 12, a helium plasma component 11, a biopsy punch 16, and an NIL component 14. Additional components may also be included in the system, which will be discussed below. One or more elements of the invention may be used or shared by one or more components of the system, without limitation.

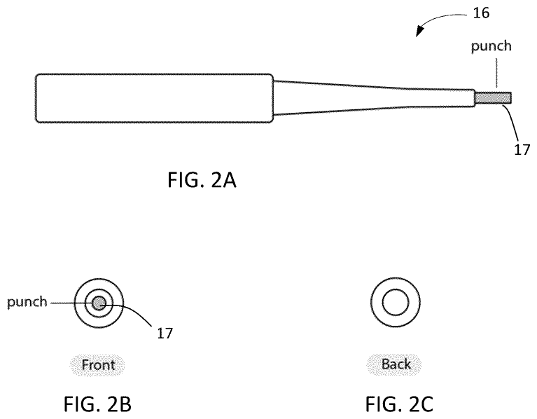

As shown in FIG. 2, a biopsy punch 16 can be used to create an incision in the skin. The biopsy punch can comprise medical grade stainless steel to reduce infection. In a further aspect, the biopsy punch of two millimeters at the incision tip 17 may be created to accommodate instrumentation of similar size. By utilizing a small biopsy punch, the resulting incision may be left open to heal naturally with zero or a negligible amount of scarring, advantageously not requiring stiches or other surgical sutures. In other examples, a biopsy punch may be created with an incision tip 17 that is 0.1, 0.2, 0.5, 1, 1.5, 2, 2.5, 3, 3.5, 4, or 5 mm. A person of skill in the art will appreciate the biopsy punch may be created with yet other alternative sizes, which may accommodate for instrumentation that may be used.

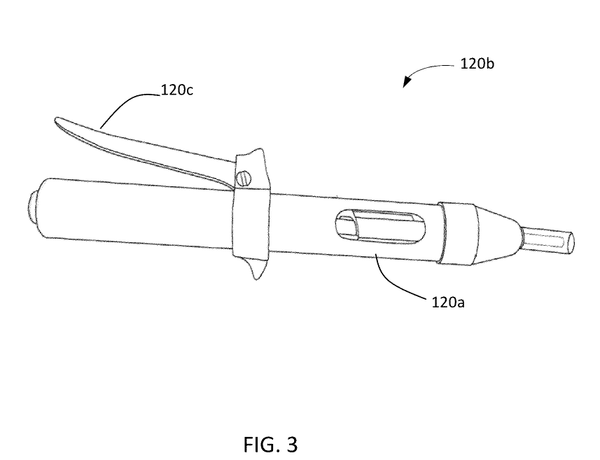

In a further aspect, as depicted in FIG. 3, the system 10 can comprise a numbing component 12. The numbing component 12 may include an anesthetic device 120b. The anesthetic device 120b may be used to reduce sensation in a localized area where the helium plasma component and/or NIL component may enter the body of the patient. By using a localized numbing process, fat removal may be performed while a patient is in an awakened state. The anesthetic device 120b of the numbing component may be a jet injector 120a as shown in FIG. 3. The jet injector 120a may propel an anesthetic into the skin of a patient using a high-pressure stream of air injected into the pores of the patient's skin. This propelled substance may be received by the subcutaneous tissue. The jet injector 120a may inject a substance into the skin of a patient without the insertion of a needle, resulting in reduced pain for the patient. The jet injection can be actuated by squeezing the lever 120c. Application of the anesthetic by jet injection is advantageous over being received by needle injection because a substance introduced into the skin using a jet injection may be more quickly dispersed throughout a localized area of the body and will not need to be diffused from an initial pool-like area caused by local needle injection.

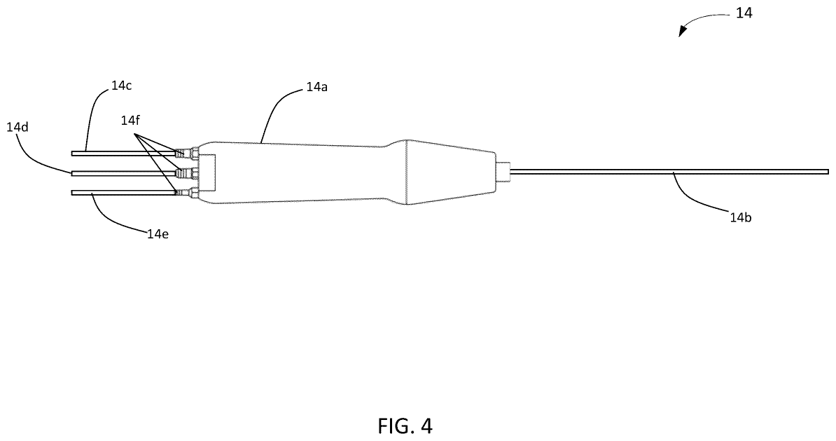

Referring to FIG. 4, the nutational infrasonic liposculpture (NIL) system 14 generally includes at least a source of nutational motion 14a, an NIL cannula 14b, and source of suction. Nutational movement would be understood by a person of ordinary skill in the art as rotational movement having a periodic variation in the inclination of the axis rotation. The source of nutational motion may include a motor, pneumatic device, or other source of rotating and/or oscillating motion. The shaft may be the NIL cannula 14b. For example, the NIL component can comprise the Euromi EVAMATIC® handpiece.

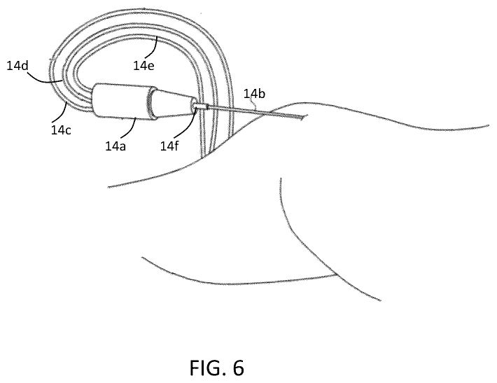

A source of suction may be included by or operatively connected to the NIL component 14. More specifically, according to at least one embodiment, the source of suction may be connected in the NIL component 14 to create suction in an NIL cannula 14b. Fat may be drawn through the NIL cannula 14b, due in part to the suction, with the received fat being expelled through a shared or dedicated output. As depicted in FIG. 6, the NIL 14b can be inserted under the skin. In one aspect, the NIL component 14 may have a suction input 14c and suction output 14d. In this embodiment, the NIL component 14 may include an additional output 14e, by which fat drawn through the NIL cannula 14b may be removed from the system and optionally harvested. In another embodiment, connectors 14f at outputs 14c, 14d, and 14e can be combined into a single connector with single output, wherein suction and harvesting and/or removal of fat cells is within the same tube. The suctioned/harvested fat may be high quality fat, having minimal debris and capable of being transferred to other areas of the body. Those of skill in the art will appreciate additional embodiments of the NIL component, which are intended to be included within the scope of this disclosure. Additionally, the NIL component may include cannulae with diameters ranging from 2.7 to 4.0 mm. In additional embodiments, cannulae may include diameters of about 0.2, 0.5, 0.7, 1, 1.2, 1.5, 1.7, 2, 2.2, 2.5, 2.7, 3, 3.2, 3.5, 3.7, 4, 4.2, 4.5, or 5 mm.

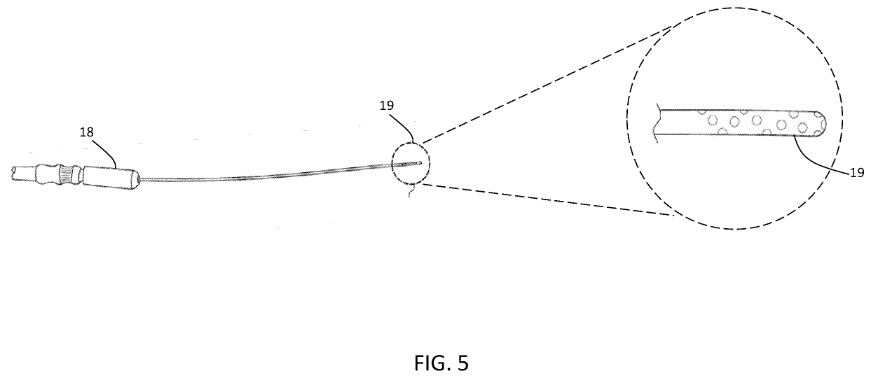

In a further aspect, as depicted in FIG. 5, a tumescent cannula 18 can be used to inject tumescent fluid into the incision made by the biopsy punch 16. The tumescent fluid can be used to help liquify the fat, making the extraction via the nutational motion of the NIL component 14 more efficient and effective. In yet a further aspect, tumescent cannula can be a local anesthetic, such as lidocaine, to the subcutaneous tissue. The anesthetic may also include epinephrine, which may affect operation of the nerves around the area receiving the anesthetic. Additionally, epinephrine may be included with lidocaine to prolong the action of the anesthesia and affect the perception of pain by the nervous system of the patient. Additionally, nitrous oxide may be provided to a patient to calm his or her nerves during a procedure. Nitrous oxide may be provided to supplement or substitute administration of other anesthetics. In a further aspect, the tumescent cannula 18 may be an 18-gauge garden spray cannula 19 through which a fluid may be passed. In other examples, the tumescent cannula may be 7, 8, 9, 10, 11, 12, 13, 14, 15, 16, 17, 18, 19, 20, 21, 22, 22s, 23, 24, 25, 26, 26s, 27, 28, 29, 30, 31, 32, 33, or 34 gauge, as will be appreciated by a person of skill in the art.

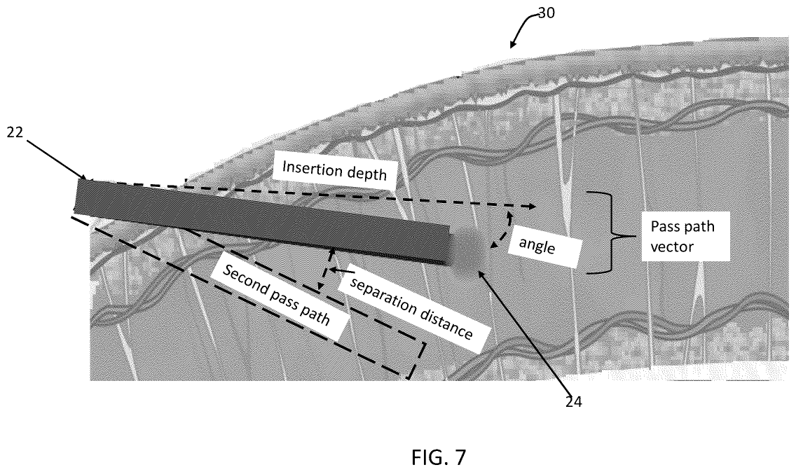

As depicted in FIG. 7, the helium plasma component can be used to tighten the skin 30 after the fat has been sucked out. During usage, a volume of gas can be injected under the skin. In one aspect the gas can be cold helium, wherein the gas helps to maintain the temperature of the skin and tightening process. The helium plasma component can be a plasma cannula 22 that is inserted into the same biopsy punch incision. The helium plasma component can be activated by providing a radio frequency pulse to the cold gas, generating a plasma stream 24. The heat from the plasma stream causes the collagen under the skin to tighten. During operation, the helium plasma cannula 22 can be inserted in the hole along a pass path. A pass path can comprise a spatial vector having a direction and distance, wherein the helium plasma cannula 22 is inserted under the skin at an angle (direction) and an insertion depth (distance).

In comparison to other body contouring procedures, the disclosed helium plasma NIL addresses advantages that surpass the results of the other procedures. The results of using helium plasma NIL can yield a stronger tightening of skin than laser supported NIL, which yields a better aesthetic result. To achieve the results, the current disclosure also employs safter operational parameters to differentiate from other systems and procedures that may also implement helium plasma skin tightening.

For example, the current method may implement an operative setting of the helium plasma at 60 to 70 percent of the standard power of 40 watts recommended for use by the manufacturers of the Renuvion helium plasma device. That is, the operative power settings would be about 24 to 28 watts instead of 40 watts. The exemplary implementation may also use 1-2 liters of gas per minute, instead of the contemporaneous wider range of 1-5 liters per minute recommended by the manufacturer. For skin tightening procedures, the manufacturer of helium plasma embodiments recommends repeating each pass path between 4-5 times. According to the method disclosed herein, each pass need only be repeated 2-3 times. Prior recommended operations parameters may also implement a spacing distance between pass paths of 1-2 cm. Here, the exemplary separation distance between two distinct pass paths can be at least 3 cm. Further, a pass path can also be executed at a particular rate. The pass rate can comprise the distance/time (e.g., centimeter (cm)/second(s)). Prior recommended pass rates are about 1-2 cm/s, whereas the current disclosure can employ a much higher rate of at least 3 cm/s and separation distances to be at least 3 cm.

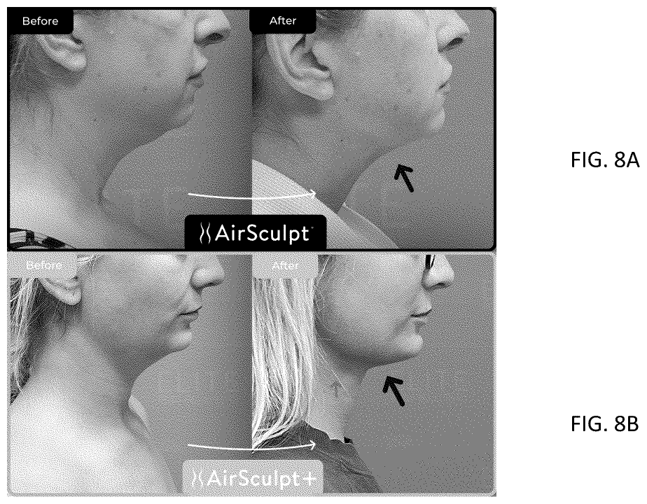

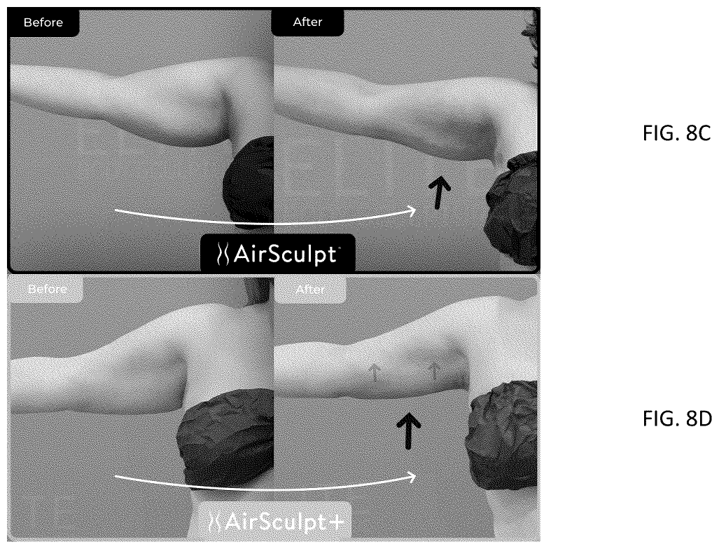

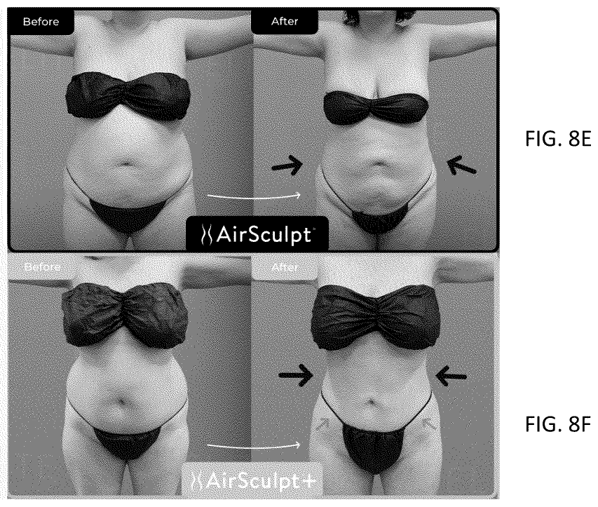

Coupling the higher pass rate, fewer passes, different gas rates, and higher separation distance with a lower power setting can make the overall procedure safer for the patient as less energy is dissipated into each volume of tissue, reducing the risk of internal burning. At the same time, this lower energy procedure has been shown to still provide improved skin tightening results relative to laser skin tightening procedures previously paired with NIL body sculpting procedures. Further, the lower energy procedure yields a reduction in bruising as comparison to other treatments. As depicted in FIGS. 8A-8F, the chin, under-arm, and stomach of the respective patients exhibit a more defined shape due to the skin being tighter from using the helium plasma component using the operating parameters (labeled as AirSculpt+) instead of a laser component treatment (labeled as AirSculpt).

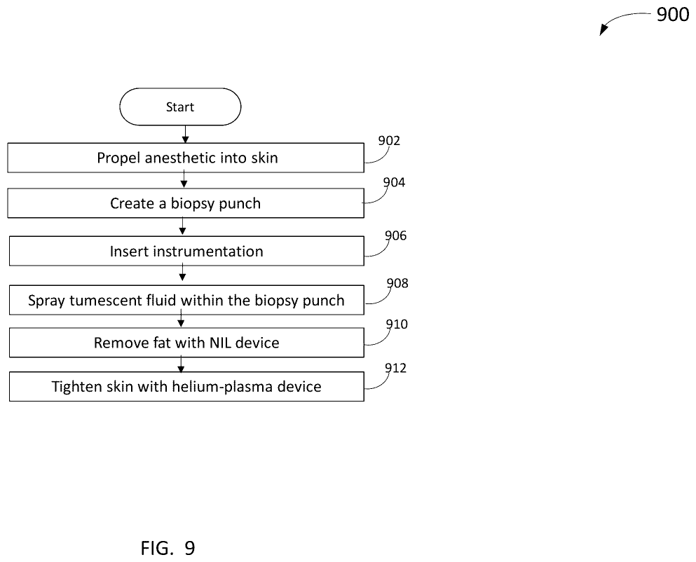

Referring to the flowchart of FIG. 9, depicting the process 900, an illustrative helium plasma NIL method may be performed using a helium plasma NIL system. Those of skill in the art will appreciate the example of process 900 to clearly illustrate an embodiment of the present invention. Skilled artisans, after having the benefit of this disclosure, will appreciate additional embodiments that would be considered within the scope and spirit of the present invention, and which are intended to be included by this disclosure. Starting at step 902, an anesthetic may be propelled or otherwise introduced into the skin. The anesthetic may be a local anesthetic, such as lidocaine and/or epinephrine. In one aspect, the anesthetic can be provided to the patient using a jet injector. The anesthetic may raise a wheal of numbness around the area through which anesthetic is dispersed. The process 900 at step 904 can include creating a biopsy punch in the skin of the patient, through which instrumentation may enter the body of the patient.

Instrumentation may be inserted into the biopsy punch at step 906. For example, a blunt tumescent cannula may be inserted into the biopsy punch. The cannula, or other instrumentation, may be configured with a size respective to the fat that may be removed. At step 908, the process 900 can include a tumescent fluid that may then be sprayed within the patient. More specifically, without limitation, the tumescent fluid may be sprayed within, or adjacent to, the fat cell of the patient near to the area from which fat may be removed. For example, the tumescent fluid may be sprayed into the patient using a hydrogen potassium (H⁺/K⁺ ATPase) surgical tumescent pump. The tumescent fluid may cause a tissue, for example fatty tissue, to become tumescent. Preferably, the tissue may become evenly tumescent. At step 910, the NIL component may be used to remove the fat cells from the patient. The NIL component may be, for example, an embodiment previously discussed in this disclosure.

The NIL cannulae included in the NIL component may be manipulated using nutation, which may increase efficiency of fat removal from the patient. Through the combination of nutational movement and suction, the NIL component may efficiently and effectively remove the fat from the patient that has been made tumescent by the steps above. At step 912, the helium plasma component can be inserted into the hole created by the biopsy punch. The helium plasma component can introduce gas (such as helium) into the cavity under the skin which is converted to a plasma. In a further aspect, a gas introduction rate of 1.5 to 2 liters per minute can be used for each pass. In one aspect, the plasma can deliver between about 24-28 watts of power. In a further aspect, a second punch hole can be inserted into the skin to provide a secondary means for allowing gas to exit the body cavity.

In a further aspect of the present invention, the fat cells extracted via the methods discussed above may be harvested. The harvested fat may be high quality, having minimal or no debris content. Fat cells that have been harvested may be transferred to a different location of a patient's body. For example, fat cells may be removed from a first location of the body using the helium plasma NIL system, via a method discussed above, and harvested. These harvested fat cells can then be transferred or implanted in a second location of the body.

As a specific example, provided without limitation, fat cells may be removed from the waist and abdominal locations of a patient. These fat cells could then be transferred to the breast location of the patient, providing a natural breast augmentation while advantageously using the patient's own tissue and not requiring the introduction of foreign objects or substances into the patient's body.

To increase a likelihood of acceptance for the transferred fat cells by the body, one or more stem cells may be transferred and/or implanted along with the fat cell. Stem cells are biological cells that can divide and differentiate into various cell types. Stem cells included with the transferred fat cells may act as progenitor cells and increase the likelihood that the body accepts a transferred fat cell. Fat cells that are accepted by the body may be anchored to the new location with assistance of the stem cells. Skilled artisans will appreciate stem cells and biological details relating to the same.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A system for performing a liposculpture procedure, the system comprising:
a biopsy punch configured to create one biopsy punch opening in the skin of a patient at a location;
a tumescent cannula for injecting a tumescent fluid into a body of the patient, wherein the tumescent cannula is insertable into the body of the patient through the one biopsy punch opening;
a nutational infrasonic liposculpture (NIL) component, the NIL component further comprising a source of nutational motion and a source of suction;
at least one NIL cannula; and
a helium plasma component comprising a plasma cannula, which can be coupled to a helium gas supply;
wherein the NIL component is attached to the at least one NIL cannula,
wherein the source of suction creates a suction through the at least one NIL cannula when the NIL cannula is inserted into the body while the NIL cannula is driven with nutational movement within the body to remove fat proximate the location of the body, and
wherein the helium plasma component supplies a gas into the body and generates a plasma stream to heat sections of tissue proximate the location;
and wherein each of the tumescent cannula, the at least one NIL cannula, and helium plasma cannula pass through the same one biopsy punch opening.

2. The system of claim 1, wherein the helium plasma component operates at a power setting between about 24-28 watts.

3. The system of claim 1, wherein the tumescent cannula is a garden spray cannula to disperse the tumescent fluid or an anesthetic fluid.

4. The system of claim 1, further comprising a jet injector to propel an anesthetic into the skin of the patient at the location.

5. A method, comprising:
creating a first biopsy punch opening in the skin of a body within a wheal of numbness;
spraying a tumescent fluid within a body of the patient via a tumescent cannula inserted into the body through the biopsy punch opening;
extracting fat cells through the biopsy punch opening via a nutational infrasonic liposculpture (NIL) cannula, configured to be driven with nutational movement within the body; and
inserting a helium plasma component into the first biopsy punch opening along a first pass path to treat the skin adjacent thereto with a plasma stream.

6. The method of claim 5, wherein the helium plasma component is operated at a power setting between about 24-28 watts.

7. The method of claim 5, wherein inserting the helium plasma component into the first biopsy punch is at a rate of at least 3 cm/s.

8. The method of claim 5, further comprising inserting the helium plasma component along a second pass path, wherein a separation distance between the first pass and the second pass path is about 3 cm.

9. The method of claim 5, further wherein tumescent fluid further comprises an anesthetic.

10. The method of claim 7, wherein inserting the NIL cannula comprises inserting the NIL cannula while the NIL cannula is attached to an NIL component that causes the nutational movement of the NIL cannula.
